(19) United States
(12) Patent Application Publication
(10) Pub. No.: US 2021/0342947 A1
(43) Pub. Date: Nov. 4, 2021

(54) COMPUTER VISION-BASED ASSESSMENT OF INSURANCE CLAIMS

(71) Applicant: Dr. Opinion, Inc., San Leandro, CA (US)

(72) Inventors: Daniel Martins Takabayashi, Pleasanton, CA (US); Danilo Nunes dos Santos, Atibaia (BR); Sung Joon Park, San Leandro, CA (US); Willian Leite, Pleasanton, CA (US)

(21) Appl. No.: 16/866,503

(22) Filed: May 4, 2020

(51) Int. Cl. G06Q 40/08; G16H 30/20

(52) U.S. Cl. CPC G06Q 40/08; G16H 30/20

(57) ABSTRACT

Described are systems and methods for assessing a dental insurance claim. The system receives a dental image and dental information for a dental claim, with the dental image and dental information being associated with a patient. The system then determines a plurality of features of the dental image, and detects anomalies based on the features of the dental image. Next, the system compares the dental information of the patient with the plurality of features of the dental image associated with the patient. Finally, the system generates a claim summary based on the comparison of the dental information with the plurality of features of the dental image.

### FIELD OF THE INVENTION

The present invention relates generally to insurance claim adjustment, and more particularly, to systems and methods for providing computer vision-based assessment of insurance claims.

### BACKGROUND

Within the insurance industry, in order to prevent potential misrepresentation and abuse of the claims process, health insurance companies require dental offices to provide a claim form along with supporting documents (e.g., “attachments”) for the claim. For dental insurance claims in particular, images, such as x-ray images or “radiographs” of treated patients are required to show the existing pathologies and/or the treatment provided. This is an important process to detect claim mistakes, abuse, and fraud, or otherwise faulty claims during the process.

A claim adjuster or claim adjudicator, i.e., an individual who analyzes and assess the claim and supporting documents, can typically range from an entry-level office worker to a specialist such as a dentist or a radiologist. The claim adjuster traditionally reviews the submitted forms and images manually. The claim adjuster may accept or deny the claim based on any discrepancies between the information provided in the claims and the features shown in the images.

This manual method of claim adjudication is significantly time consuming, resource-intensive, inefficient, and prone to human errors and inconsistency. A typical insurance company (i.e., insurer) has millions of insurees, which may result in hundreds or thousands of images, charts, and forms to be processed daily. With such a large volume of data and limited analysis time, the claim adjusters may easily get fatigued and time-pressured, resulting in inconsistent and inaccurate assessment.

Moreover, human labor is typically expensive. For this reason, health insurance companies may hire inexperienced adjudicators or skip some of the image validation processes. Insurance companies often go through these processes only when the claimed procedures are uncommon or suspicious on their face. Some insurance companies may opt to process only portions of the images as routine samples.

Thus, there is a need in the field of insurance claim adjustment to create a new and useful system and method for assessing insurance claims, such as dental insurance claims, in an automated or semi-automated way. The source of the problem, as discovered by the inventors, is a lack of computer-vision based processes for claim image analysis and insurance claim adjustment which need not rely upon expensive, error-prone human labor.

### SUMMARY

The invention overcomes the existing problems by automating part or all of the claim adjustment and adjudication process. By employing computer vision processes built on artificial intelligence models such as, e.g., deep neural networks, the assessment of dental claims can be efficiently provided, allowing the companies to consistently process many claims and make informed decisions on them. Such models can utilize image data as well as non-image information for training and evaluation in an effective way. A well-trained system can, in many cases, detect pathologies which human eyes have trouble detecting or do not have the capacity to detect at all. This is especially true when considering many claims adjusters lack the skill and training to detect such pathologies. By developing such techniques, the human errors in conducting claim adjudication can be reduced, and the health insurance system can be improved overall.

One embodiment relates to systems and methods for assessing a dental claim. The system first receives a dental image and dental information associated with a patient. Thereafter, the system determines features of the dental image, then detects anomalies based on at least the features of the dental image. Next, the system compares the dental information of the patient with the features of the dental image, and generates a claim summary based on at least the comparison.

In some embodiments, after receiving the dental image, the system detects an image type of the dental image (e.g., bitewings, panoramics, etc). The system then determines dental image information based on the image type, and determines one or more features associated with the dental image.

Further areas of applicability of the present disclosure will become apparent from the detailed description, the claims and the drawings. The detailed description and specific examples are intended for illustration only and are not intended to limit the scope of the disclosure.

### BRIEF DESCRIPTION OF THE DRAWINGS

The present disclosure will become better understood from the detailed description and the drawings, wherein:

FIG. 1A is a diagram illustrating an exemplary environment in which some embodiments may operate.

FIG. 1B is a diagram illustrating an exemplary computer system that may execute instructions to perform some of the methods herein.

FIG. 2A is a flow chart illustrating an exemplary method that may be performed in some embodiments.

FIG. 2B is a flow chart illustrating additional steps that may be performed in accordance with some embodiments.

FIG. 3A is a diagram illustrating one example embodiment 300 of model components within a computer vision-based claim assessment process, in accordance with some embodiments.

FIG. 3B is a diagram illustrating another example embodiment 320 of model components within a computer vision-based claim assessment process, in accordance with some embodiments.

FIG. 3C is a diagram illustrating one example embodiment 340 of a process check performed in accordance with some embodiments.

FIG. 4 is a diagram illustrating an exemplary computer that may perform processing in some embodiments.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

In some embodiments, the systems and methods function to automate the claim adjudication process, in whole or in part. The system receives input information relating to a dental claim, and generates a claim summary or report, i.e., a claim validation summary as output based on at least the input information from, e.g., claim forms, dental images (e.g., x-ray images or radiographs), dental charts, doctor's notes, prescriptions, and if dental procedures are performed, pre- and post-operative images, or any other suitable information related to a claim.

In some embodiments, the system includes a set of model components which are assigned with individual tasks. The model components relate to one or more artificial intelligence models for analyzing the input information, such as machine learning models, deep learning models, and/or computer vision models. The model components are configured to perform claim analysis tasks such as processing images, classifying images, transforming free-text to structured data, and more. In some embodiments, the interactions and activities of the model components are determined by a set of rules (e.g., business rules, empirical rules, technical/specialized rules, or any other suitable rules). In some embodiments, the rules can be customized based on the nature and purpose of the use and the user's (e.g., client's) preference. In some embodiments, the rules are subject to change depending on use cases or situations.

The outcomes of the model components are consolidated and summarized to generate and produce the claim summary. In general, the claim summary provides the possibility of misrepresentation of the claims (e.g., mistakes, fraud, human errors) and overtreatment. Specifically, the summary may address whether the images belong to the identified patient, whether the procedures shown in the images match the standard procedures for identified pathologies, and more.

The automated or semi-automated claim adjustment procedures herein may serve to benefit in various situations and use cases. For example, they may assist a dental professional in making a diagnosis of the patient's condition; provide a “second opinion” that may confirm the dental professional's findings or point to ambiguities that call for a more detail analysis; provide education as a learning tool for continuing education of dental professionals; estimate the quality of the dental practice; or a myriad number of additional benefits and uses.

I. Exemplary Environments

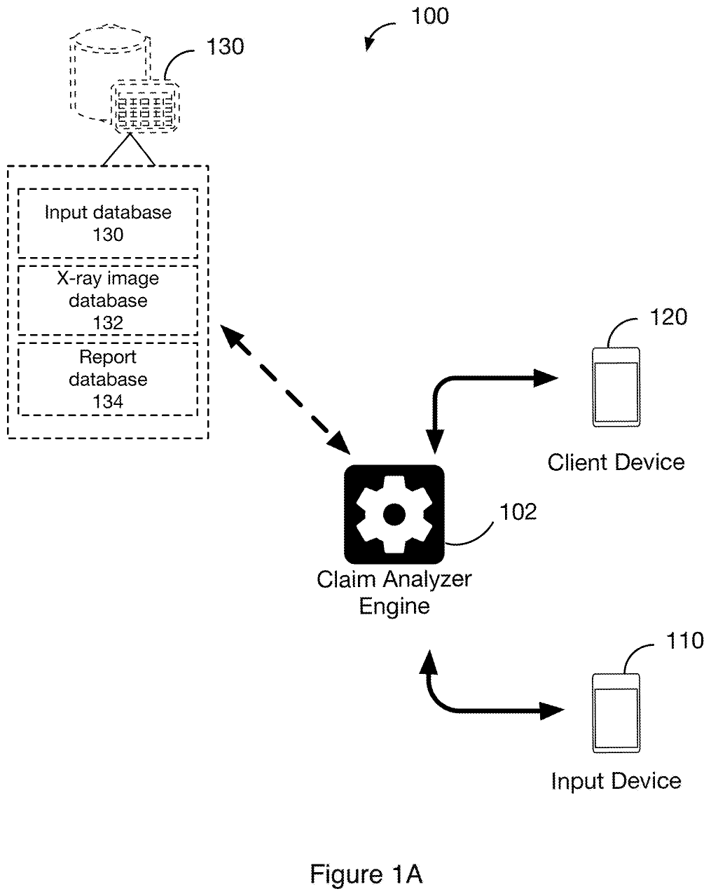

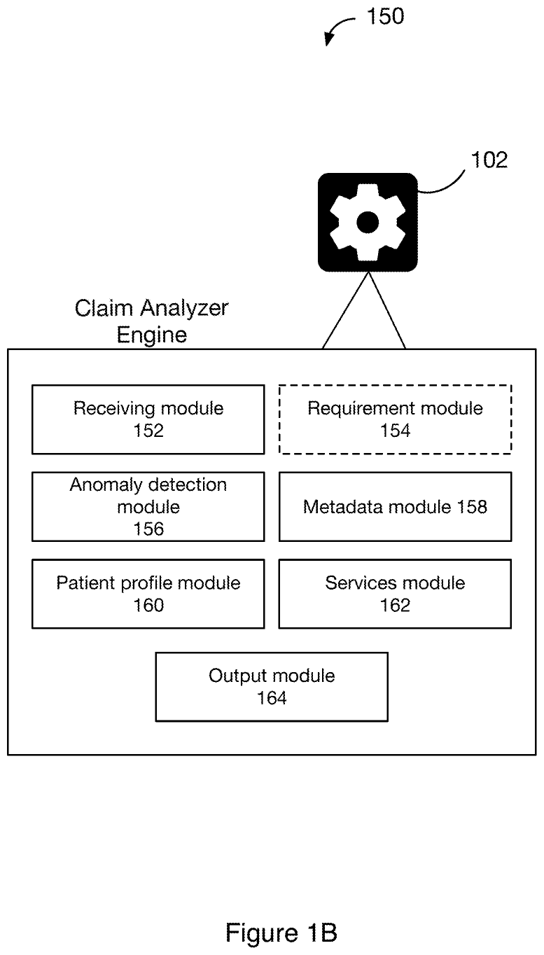

FIG. 1A is a diagram illustrating an exemplary environment in which some embodiments may operate. In the exemplary environment 100, a client device 120 and an input device 110 are connected to a claim analyzer engine 102. The claim analyzer engine 102 is optionally connected to one or more database(s), including an input database 130, x-ray image database 132, and/or a report database 134. One or more of the databases may be combined or split into multiple databases. The input device and/or client device in this environment may be computers.

The exemplary environment 100 is illustrated with only one client device, input device, and claim analyzer engine for simplicity, though in practice there may be more or fewer client devices, input devices, and/or claim analyzer engines. In some embodiments, the client device, input device, and/or claim analyzer engine may be part of the same computer or device.

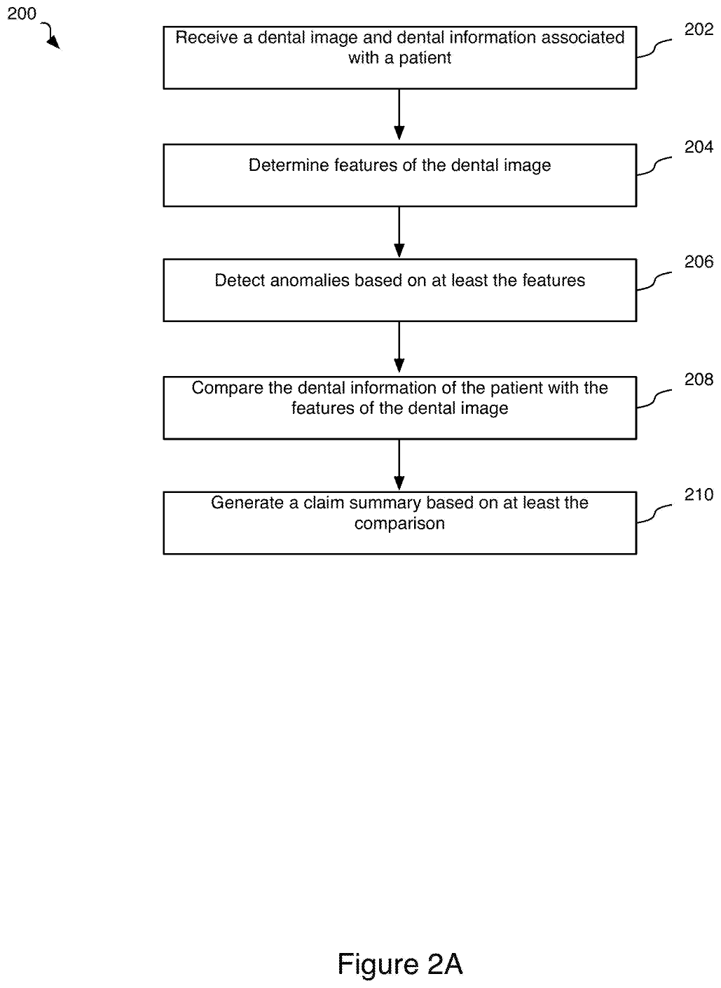

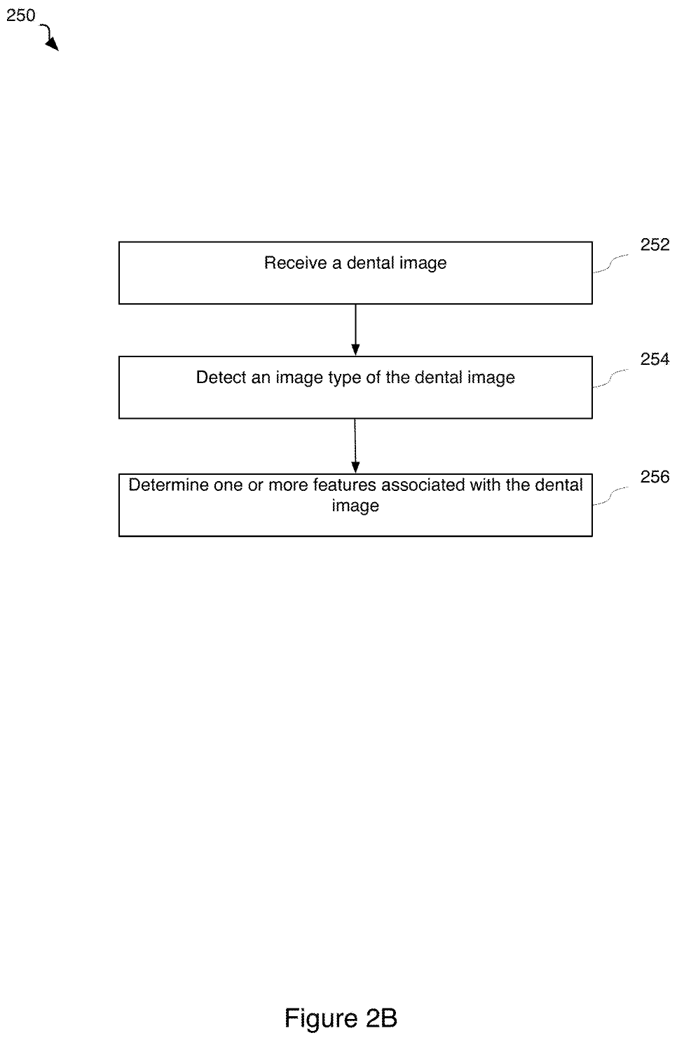

In an embodiment, the claim analyzer engine 102 may perform the method 200 (FIG. 2A) or other method herein and, as a result, provide claim assessment for dental claims in an automated or semi-automated fashion. In some embodiments, this may be accomplished via communication with the client device, input device, or other device(s) over a network between the client device 120, input device 110, or other device(s) and an application server or some other network server. In some embodiments, the claim analyzer engine 102 is an application hosted on a computer or similar device, or is itself a computer or similar device configured to host an application to perform some of the methods and embodiments herein.

Input device 110 is a device that sends input information (e.g., images, image-related data, patient data, or any other suitable information) to the claim analyzer engine 102. In some embodiments, the input device 110 receives notification or confirmation of receipt in response to sending the input information. In some embodiments, input device 110 is a computing device capable of hosting and executing one or more applications or other programs capable of sending and/or receiving information. In some embodiments, input device 110 may be a computer desktop or laptop, mobile phone, virtual assistant, virtual reality or augmented reality device, wearable, or any other suitable device capable of sending and receiving information. In some embodiments, the claim analyzer engine 102 may be hosted in whole or in part as an application executed on the input device 110.

Client device 120 is a device that receives claim reports and/or other claim assessment information from the claim analyzer engine 102. In some embodiments, the client device 120 receives such information in the form of a dashboard, a set of dashboards, or other user interface elements or environments. In some embodiments, client device 120 is a computing device capable of hosting and executing one or more applications or other programs capable of sending and/or receiving information. In some embodiments, the client device 120 may be a computer desktop or laptop, mobile phone, virtual assistant, virtual reality or augmented reality device, wearable, or any other suitable device capable of sending and receiving information. In some embodiments, the claim analyzer engine 102 may be hosted in whole or in part as an application executed on the client device 120. In some embodiments, the input device 110 and client device 120 may be the same device.

Optional database(s) including one or more of an input database 130, x-ray image database 132, and/or a report database 134 function to store and/or maintain, respectively, input information received from input device 110, x-ray image or other image information received from input device 110, and/or claim reports generated based on at least the claim assessment. The optional database(s) may also store and/or maintain any other suitable information for the claim analyzer engine 102 to perform elements of the methods and systems herein. In some embodiments, the optional database(s) can be queried by one or more components of system 100 (e.g., by the claim analyzer engine 102), and specific stored data in the database(s) can be retrieved.

FIG. 1B is a diagram illustrating an exemplary computer system 150 with software modules that may execute some of the functionality described herein.

Receiving module 152 functions to receive input information from one or more sources, such as an input device 110 which sends the input information to the claim analyzer engine 102. Input information can include, e.g., claim forms, x-ray or other images, dental charts, doctor's notes, prescriptions, pre- and post-operative images, or any other information relating to a claim.

Requirement module 154 functions to perform a requirements check with respect to the received input information.

Anomaly detection module 156 functions to detect anomalies, i.e., outliers, within the input information based on existing patterns of fraud.

Metadata module 158 functions to perform analysis and processing of metadata within the input information that is related to the dental claim.

Patient profile module 160 functions to perform image comparison and matching processes with respect to patient profile data.

Services module 162 functions to perform analysis and assessment of services (e.g., treatments and procedures) provided by the dentist or specialist.

Output module 164 functions to provide an output as a result of the analysis and processing performed, such as, e.g., a claim summary. Output module 164 provides this output to one or more external devices, such as, e.g., the client device 120.

The above modules and their functions will be described in further detail in relation to an exemplary method below.

II. Exemplary Method

FIG. 2A is a flow chart illustrating an exemplary method that may be performed in some embodiments.

At step 202, the system receives dental images and dental information associated with a patient. In some embodiments, a received dental image, such as a dental radiographic image, is received as input information from an input device 110 or other external source of input information. In some embodiments, the system does not capture or generate dental images, but rather uses images received by or provided by an input device, imaging software hosted on a device, or other external source. Such images can include, e.g., bitewing x-rays, periapical x-rays, panoramic x-rays, intra-oral images, computed tomography (“CT”) scans (“CT-scans”) coming from a CT scanner, or photos or screenshots of any one of these. In some embodiments, if dental procedures were performed as part of the submitted claim, then images may include pre- and/or post-operative images.

In some embodiments, dental information can include claim forms. Claim forms which may be received as input information can include one or more of: insurance company information (e.g., company name and address), policyholder or subscription information, patient information (e.g., name, date of birth, address, gender, relationship to policyholder, or other suitable or relevant patient information), a record of services provided (e.g., date, tooth numbers, tooth surfaces, procedure codes, descriptions, and/or fees), treating dentist information (e.g., name, address, and license number), or any other suitable claim forms.

In some embodiments, dental information can include medical records. Medical records which may be received as input information can include, e.g., dental charts, doctor's notes, prescriptions, or any other suitable or relevant medical records.

In some embodiments, the system optionally includes the step of performing a requirements check. The requirements check functions to verify that basic requirements for the system with respect to the received input information are satisfied. In some embodiments, the requirements check can include determining whether the required fields in the claim form are correct and completed, images are provided as requested, services provided are supported by correct image type (e.g., if an implant is provided, an insurance company will likely require both pre- and post-operative bitewing images), or any other suitable requirements. In some embodiments, a user or client can define, modify, insert or delete requirements, or otherwise customize the requirements check process to their needs.

At step 204, the system determines features of the dental image. In some embodiments, features are relevant characteristics, parameters, or criteria which factor into claim assessment or adjustment. In some embodiments, the features are predicted, wherein the output prediction is from a machine learning model or other artificial intelligence model trained on a set of labeled data. Determining and/or predicting the features can involve computer vision technology, such as classification techniques, or other artificial intelligence processes or models. In some embodiments, the techniques can additionally or alternatively include object detection, object tracking, segmentation, regression, siamese neural network, and other known feature determination techniques. In some embodiments, the feature determination process involves determining appropriate features from images based on a corresponding appropriate image type, wherein the image type is first detected as a feature from the image (via, e.g., classification technique), and then the additional appropriate features are determined based on that appropriate image type. For example, in some embodiments, a gender of the patient may be determinable with high accuracy from, e.g., panoramic and bitewing x-rays. Thus, in that instance, the patient's gender is an appropriate feature which can be determined from the corresponding appropriate image type (panoramic or bitewing x-rays). Similarly, in some embodiments and examples, a dental procedure may be determinable from bitewing x-rays.

As mentioned above, in some embodiments, the system detects an image type for the dental image as part of the feature determination process. In some embodiments, this detection is performed using the classification techniques. In some embodiments, the classification techniques employ machine learning or other artificial intelligence processes or models trained to classify images into distinct categories based on a visual appearance of each category. In some embodiments, the categories of image type can be, e.g., bitewing x-ray image, periapical x-ray image, panoramic x-ray image, intra-oral image, computed tomography image, any combination thereof, or any other suitable image type for the dental image.

In some embodiments, the system detects an object within the dental image as part of the feature determination process. In some embodiments, this detection is performed using the object detection techniques. In some embodiments, the object detection techniques employ machine learning or other artificial intelligence processes or models trained to detect objects within the image. In some embodiments, the objects detected as part of the feature determination process include, e.g., cavities, dental pathologies, specific tooth numbers, missing teeth, dental procedures (e.g., crown procedure, crown-bridge procedure, root canal, braces, fillings-amalgam, fillings-composite, or any other suitable dental procedure which can be determined from the image), bone loss, cavities intensity, and any other suitable determinable features within the image.

In some embodiments, the system detects patient anatomy within the dental image as part of the feature determination process. In some embodiments, this determination is based on the visual similarity of each anatomical feature, using siamese neural networks, convolutional neural networks or other computer vision technology. In some embodiments, the anatomical features may include, e.g., a structure, shape, color, size, and other features of oral cavity determinable within the dental images. In some embodiments, the anatomical features and other features detected (e.g., cavities, dental procedures, bone loss) may be used to determine that multiple dental images associated with a patient actually belong to that same patient. The multiple dental images could be provided by a dentist to support a single dental claim or could be gathered from past and present claims associated with the patient.

In some embodiments, the system detects the gender and the age of a patient as part of the feature determination process. In some embodiments, this determination is based on the visual similarity of each category of gender and the visual similarity of each category of age, using classification techniques or other computer vision techniques. In some embodiments, the categories of gender may include, e.g., biological male and biological female. In some embodiments, the categories of age may include, e.g., early adolescence (0˜13 years old), late adolescence (14˜20 years old), young adults (20˜40 years old), mature adults (40˜65 years old), or elderly (65˜years old).

In some embodiments, the system detects a duplicate image as part of the feature determination process. A duplicate image is an image that is similar or the same as another image that is used to support a separate dental claim. In some embodiments, this determination is based on the visual similarity between each image, using convolutional neural networks or other computer vision techniques. In some embodiments, the duplicate image may include any used image that has been distorted, modified, or manipulated to support a new claim.

In some embodiments, feature determination can additionally or alternatively include image characteristics, including, e.g., image color, image intensity, image quality, distortions within the image, an image source, or any other suitable image characteristics. In some embodiments, image characteristics such as, e.g., image source and distortions can be detected by studying the file metadata additionally or alternatively to the above-described techniques.

In some embodiments, input information for various machine learning models, for example dental information (such as, for example, information parsed from a submitted dental claim form), can include, e.g., procedures performed, fees for procedures, dentist reputation, or any other relevant features. In some embodiments, the input information can be used together with the determined features to adjudicate claims pursuant to the steps described below.

At step 206, the system detects anomalies based on at least the features of the dental image. Anomalies represent outliers which stand out from one or more pieces of data and which may conform to patterns of fraud prevalent in the insurance industry. In some embodiments, the anomalies are detected based on internal factors, i.e., factors internal to the dental image. These may be based on at least the features determined from the image in step 204. Such internal factors may include, e.g., an unusually high number of procedures or pathologies detected within the image, an unusual shape of human anatomy, and any other suitable internal factors. In some embodiments, anomaly detection can be additionally or alternatively based on factors external to the features within the image (i.e., the determined features from step 204). Such external factors may include, e.g., unusually expensive fees being charged, dentists with red flags from previous claim adjudication processes, an unusually high number of procedures as provided in the claim form, and any other suitable external factors.

At step 208, the system compares the dental information of the patient with the plurality of features of the dental image associated with the patient. In some embodiments, the system structures the text within the claims, and then analyzes the structured data by comparing it to the features within the dental images.

In some embodiments, this comparison can involve processing and analysis of metadata information by one or more models. Metadata can include, e.g., image source, image editing history, image type, image file type, image file name, file creation date, image resolution, image color, image intensity, image distortions, image modifications, or any combination thereof. Such metadata analysis can include one or more of: retrieving an image source, retrieving one or more historical instances of image editing or modification, detecting image type, extracting a histogram of colors and intensity, identifying image quality such as resolution and/or dimension data, identifying duplicate images which are the same or similar images as used in previous claims (for similar claims, the images could include used images with distortions, modifications, or other evidences of manipulation of the image).

In some embodiments, this comparison can involve one or more patient profile process and analysis tasks. These may include, for example, image-gender matching (i.e., a gender detected within the image matches the gender identified in the claim), image-age matching (i.e., an age detected within the image matches the age identified in the claim); matching of multiple images (i.e., if multiple images are provided to support a claim, all of them are determined to belong to the same patient), and/or patient-patient matching (e.g., retrieving images of the patient from his past claims, wherein the current image and the retrieved images are determined to belong to the same patient).

In some embodiments, the comparison involves processing and analysis of services provided by the dental professionals. These may include, for example, detecting pathologies within the image (e.g., cavities, gum disease), detecting procedures within the image (e.g., implants, crowns, fillings), and comparing the detected pathologies and/or procedures with those described in the claim form. In some embodiments, the system may find misrepresentation if, for example, the pathologies or procedures described in the claim do not represent those detected within the image (e.g., the procedure detected within the dental image does not match the procedure code, tooth number, and/or the tooth surface as described in the claim). In some embodiments, the system may find overtreatment if, for example, the procedure rendered by a treating doctor goes beyond the standard procedure for the existing condition (e.g., pathologies) of the patient detected within the image. The system reports any discrepancies as described in step 210.

In some embodiments, the system determines whether the dental image includes fraudulent manipulations. This determination is based on at least the image analysis and comparison steps described above. In some embodiments, determination of fraudulent manipulations can include, e.g., checking the image to see if an implant can be located in the tooth which was required to be present based on the procedures (for example, if the claim procedure was implant #10, then the system checks for implants in the tooth #10); determining if a given tooth is healthy or not, wherein if there's no need to treat a tooth because it is healthy, then overtreatment may be occurring; when two images are sent in a claim, checking that they're both of the same patient; determining that the patient in the image appears to be consistent with the patient profile information provided in the claim; detecting any editing or manipulation of the image; detecting whether the image is of someone else's image within an existing database who is not the patient; detecting whether the image is duplicative of used images; and any other suitable fraud detection approaches or determinations.

At step 210, the system generates a claim summary based on at least the comparison of the dental information with the plurality of features of the dental image. For instance, the claim summary can include, e.g., a list of fraudulent manipulations, misrepresentations, overtreatments, and/or errors found (e.g., a requirements check can reveal that the doctor's name is missing from the submitted claim, or a patient profile check can reveal no errors.) In some embodiments, the generated claim summary is provided to the client device 120 for display at a user interface or other environment. In some embodiments, the claim summary can include the submitted input images but with modifications to show bounding boxes which segment out various features of the image, such as cavities and dental procedures.

FIG. 2B is a flow chart illustrating additional optional steps that may be performed in accordance with some embodiments.

At optional step 252, the system receives a dental image, as in step 202.

At optional step 254, the system determines an image type of the dental image. The system determines this image type using feature determination techniques, such as, e.g., classification technique, as discussed above with respect to step 204 of FIG. 2A. The image type may include, e.g., panoramic, intra-oral, bitewing, or other form of image type.

At optional step 256, the system determines one or more features associated with the dental image. In some embodiments, the system determines these features by performing feature detection and/or feature determination processes. In some embodiments, the features are determined based at least in part on the detected image type, as described above with respect to step 204 of FIG. 2A.

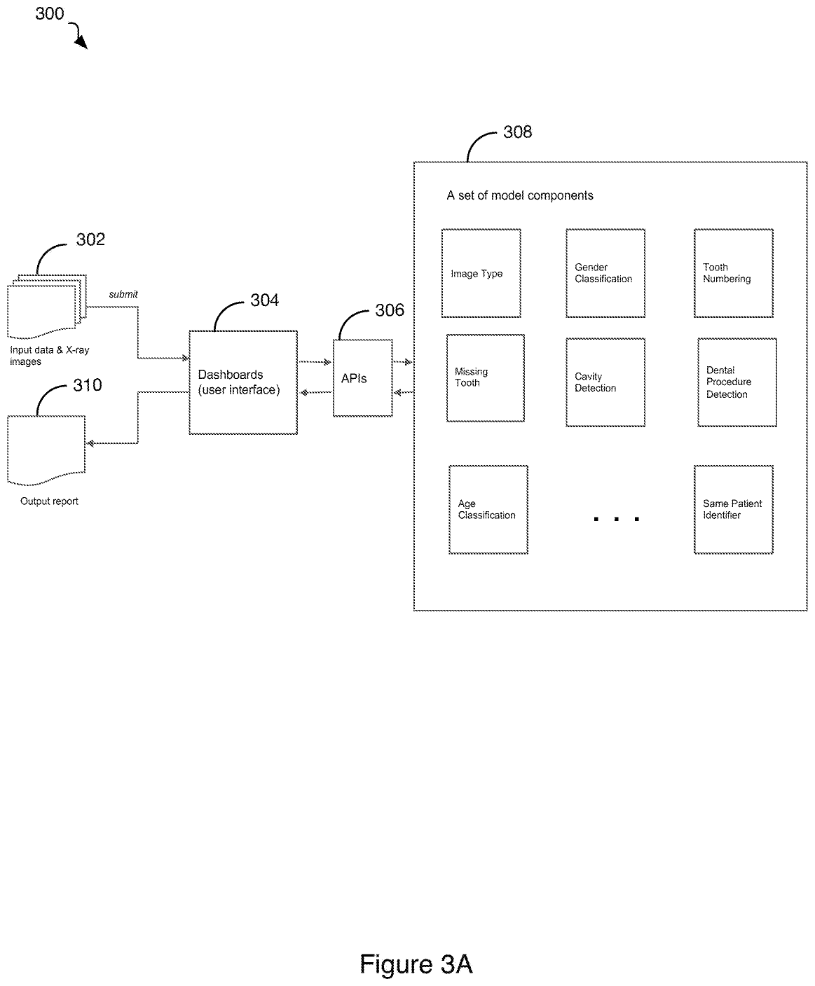

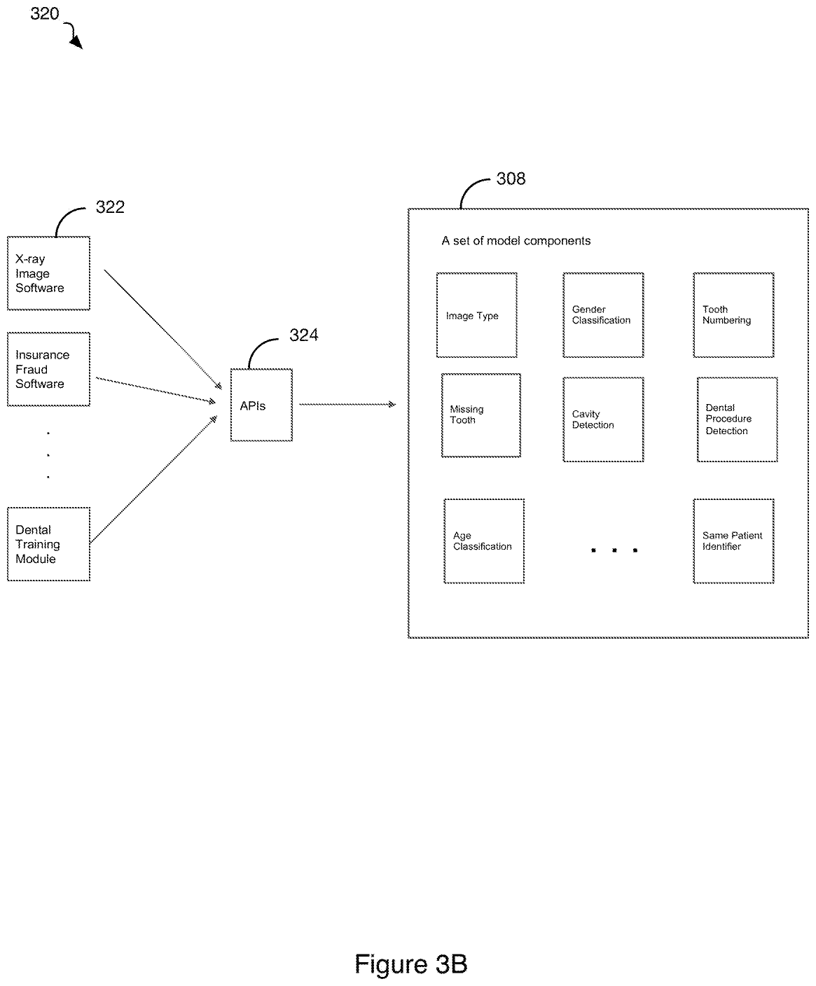

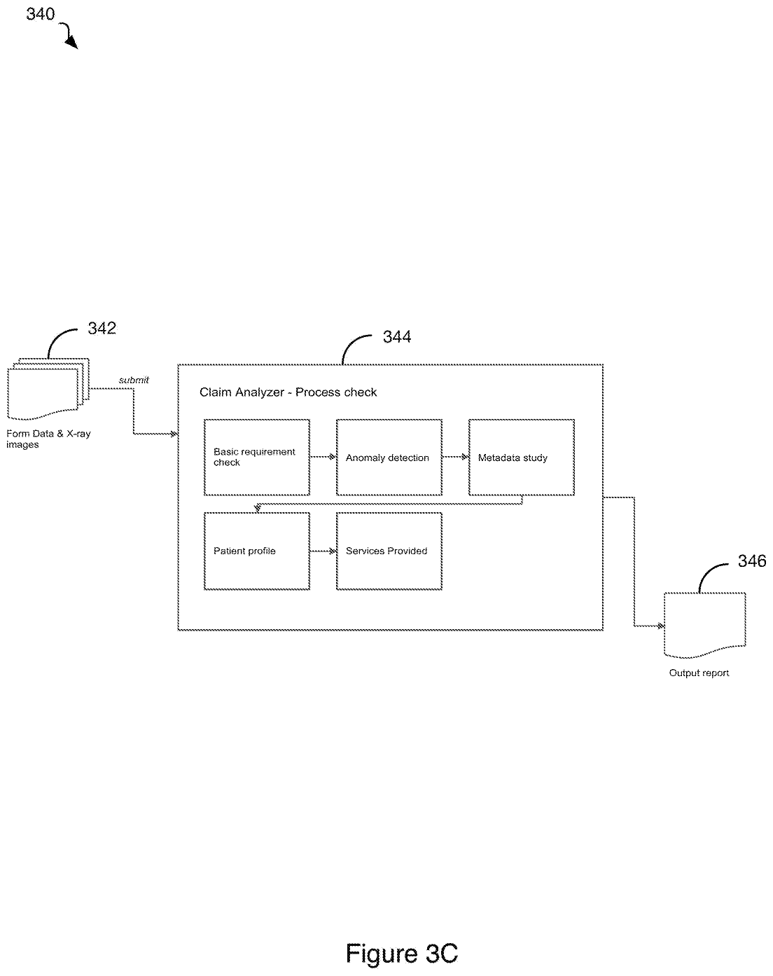

FIG. 3A is a diagram illustrating one example embodiment 300 of model components within a computer vision-based claim assessment process, in accordance with some embodiments. The diagram illustrates an example workflow for claim assessment and adjudication including one or more model components for performing claim analysis.

Input data and x-ray images 302 are submitted by an input device 110 using one or more dashboards within a user interface 304. In some embodiments, the dashboards display information relating to claim summary and other relevant information. In some embodiments, the dashboards are fully stacked to include all obtained information and all assessments for the claim, to provide the most information possible to a client (e.g., health insurance company representative). In some embodiments, the dashboards allow a user or client to receive and modify information pertaining to claims, cases, connections of cases to previous cases, and other suitable tasks.

The input information is then sent to Application Programming Interfaces (“APIs”) 306. In some embodiments, the APIs can be integrated to one or more existing pieces of software utilized by insurance companies, billing providers and/or medical billing clearinghouses. In some embodiments, the pieces of software integrating the APIs define one or more sets of rules for claim analysis and assessment.

The input information is then sent on to a set of one or more model components 308 through the APIs 306. The set of model components can perform computer vision and/or other artificial intelligence tasks. In some embodiments, the model components can vary per embodiment, and can be customized or modified in various ways. Examples of model components can include: image type detection, anomaly detection, metadata analysis, image search, gender prediction (e.g., classification of gender), age prediction (e.g., classification of age), same patient identifier (e.g., if multiple images are submitted, determining whether the images belong to the same patient or different patients), dental procedures detection (e.g., object detection including generation of bounding boxes for segmenting different procedures within an image), tooth numbering (e.g., object detection including generation of bounding boxes for tooth numbers), missing tooth detection (wherein the input can include, e.g., locations of two teeth and their distance, and the output can include, e.g., whether or not there is a missing tooth), cavity detection (e.g., object detection via a convolutional neural network or “CNN”, including generation of bounding boxes around detected cavities), bone loss detection, cavity intensity detection, unerupted tooth detection (e.g., object detection including generation of bounding boxes of unerupted teeth), wisdom tooth detection (e.g., object detection including generation of bounding boxes of wisdom teeth), and potentially many other model components which can be contemplated and used in accordance with the systems and methods herein. The outputs of each model component are then sent back to the pieces of software through the APIs 306. The software consolidates and summarizes the outputs via the set of rules and algorithms that are customizable based on the client (e.g., insurance company) preference. The consolidated summarization, or an output report 310, is displayed on the dashboards or user interface 304.

FIG. 3B is a diagram illustrating another example embodiment 320 of a computer vision-based claim assessment process, in accordance with some embodiments. The diagram illustrates a number of pieces of software 322 integrated into the systems and methods herein via a set of APIs 324 which allow the pieces of software to use and benefit from a set of model components 308. The various pieces of software 322 include, e.g., x-ray image software, insurance fraud software, a dental training module, and any other pieces of software which may be suitable. These pieces of software function to provide input information to the system, including dental images and/or dental information.

FIG. 3C is a diagram illustrating one example embodiment 340 of a process check (i.e., claim adjustment) performed in accordance with some embodiments. Form data and x-ray images 342 are received by the system as input information. The claim analyzer engine 344 then performs the process check on the input information, including, in order, a basic requirement check, anomaly detection, metadata analysis, patient profile analysis, and services provided analysis, as described above. An output report 322 in the form of a claim summary is then displayed on dashboards within a user interface of a client device.

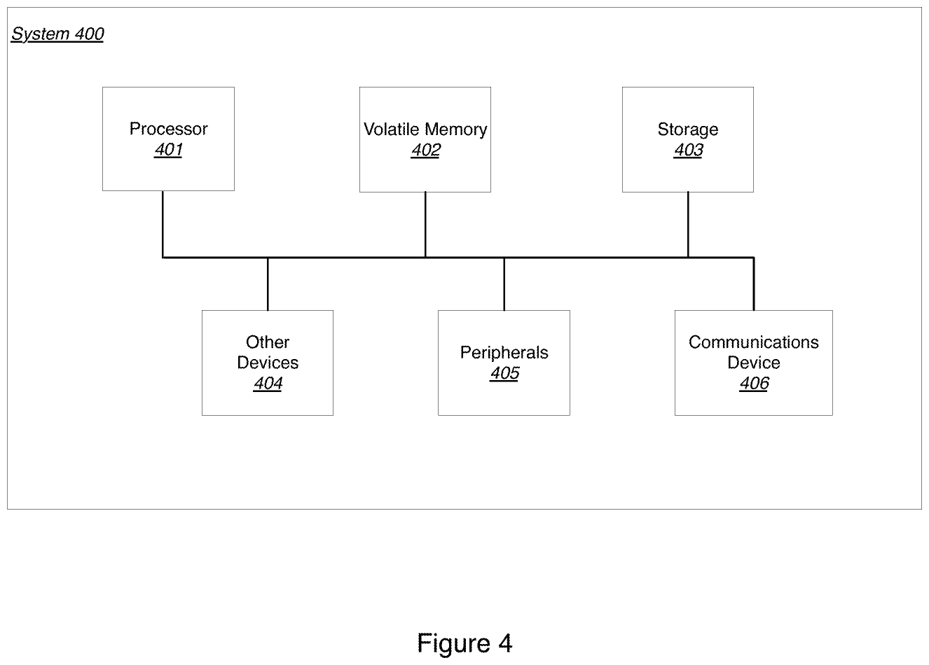

FIG. 4 is a diagram illustrating an exemplary computer that may perform processing in some embodiments. Exemplary computer 400 may perform operations consistent with some embodiments. The architecture of computer 400 is exemplary. Computers can be implemented in a variety of other ways. A wide variety of computers can be used in accordance with the embodiments herein.

Processor 401 may perform computing functions such as running computer programs. The volatile memory 402 may provide temporary storage of data for the processor 401. RAM is one kind of volatile memory. Volatile memory typically requires power to maintain its stored information. Storage 403 provides computer storage for data, instructions, and/or arbitrary information. Non-volatile memory, which can preserve data even when not powered and including disks and flash memory, is an example of storage. Storage 403 may be organized as a file system, database, or in other ways. Data, instructions, and information may be loaded from storage 403 into volatile memory 402 for processing by the processor 401.

The computer 400 may include peripherals 405. Peripherals 405 may include input peripherals such as a keyboard, mouse, trackball, video camera, microphone, and other input devices. Peripherals 405 may also include output devices such as a display. Peripherals 405 may include removable media devices such as CD-R and DVD-R recorders/players. Communications device 406 may connect the computer 100 to an external medium. For example, communications device 406 may take the form of a network adapter that provides communications to a network. A computer 400 may also include a variety of other devices 404. The various components of the computer 400 may be connected by a connection medium such as a bus, crossbar, or network.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any particular programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. A method for assessing a dental insurance claim, the method comprising:
receiving a dental image and dental information for a dental claim, wherein the dental image and dental information are associated with a patient;
determining a plurality of features of the dental image;
detecting anomalies based on at least the features of the dental image;
comparing the dental information of the patient with the plurality of features of the dental image associated with the patient; and
generating a claim summary based on at least the comparing of the dental information with the plurality of features of the dental image.

2. The method of claim 1, wherein determining the plurality of features comprises:
detecting an image type for the dental image, and
determining at least one of the plurality of features based on at least the detected image type.

3. The method of claim 1, further comprising:
determining anomalies based on at least comparing the dental information of the patient with the plurality of features of the dental image associated with the patient.

4. The method of claim 1, further comprising:
analyzing metadata information of the dental image including image source, image editing history, image type, image file type, image resolution, image color, image intensity, image distortions, image modifications, or any combination thereof.

5. The method of claim 1, wherein detecting the anomalies based on at least the features of the dental image comprises determining whether the dental image includes fraudulent manipulations.

6. The method of claim 1, wherein the image type can be bitewing x-ray image, periapical x-ray image, panoramic x-ray image, intra-oral image, computed tomography image, or any combination thereof.

7. The method of claim 1, wherein the dental information of the patient can include patient profile information including information associated with an insurance company name, insurance company address, policyholder subscription information, policyholder name, policyholder residential address, or any combination thereof.

8. The method of claim 1, wherein the dental information of the patient can include patient profile information including information associated with patient gender identification, patient age, previously claimed images associated with the patient, or any combination thereof.

9. The method of claim 1, further comprising:
matching the features of the dental image associated with the patient with dental information associated with an additional patient.

10. The method of claim 1, further comprising:
matching the features of the dental image associated with the patient with features of an additional dental image associated with the patient or an additional patient.

11. The method of claim 1, wherein the dental information of the patient can include records of services provided comprising information related to date of service, tooth numbers services, tooth surface, procedure codes, service descriptions and notes, fee information, or a combination thereof.

12. A method for analyzing a dental image, the method comprising:
receiving a dental image, wherein the dental image is associated with a patient;
determining an image type of the dental image;
determining one or more features associated with the dental image, wherein the features comprise at least one of:
identifying a plurality of teeth and tooth numbering associated with each tooth of the plurality of teeth of the patient,
detecting missing teeth associated with the patient,
identifying a gender of the patient,
identifying an age of the patient,
identifying one or more historical dental procedures provided to the patient, and
identifying one or more pathologies associated with the patient.

13. A non-transitory computer-readable medium containing instructions for assessing a dental claim, comprising:
instructions for receiving a dental image and dental information for a dental claim, wherein the dental image and dental information are associated with a patient;
instructions for determining a plurality of features of the dental image;
instructions for detecting anomalies based on at least the features of the dental image;
instructions for comparing the dental information of the patient with the plurality of features of the dental image associated with the patient; and
instructions for generating a claim summary based on at least the comparing of the dental information with the plurality of features of the dental image.

14. The non-transitory computer-readable medium of claim 13, further comprising:
instructions for detecting an image type for the dental image, and
Instructions for determining at least one of the plurality of features based on the detected image type.

15. The non-transitory computer-readable medium of claim 13, further comprising:
instructions for determining anomalies based on comparing the dental information of the patient with the plurality of features of the dental image associated with the patient.

16. The non-transitory computer-readable medium of claim 13, further comprising:
instructions for analyzing metadata information of the dental image including image source, image editing history, image type, image file type, image resolution, image color, image intensity, image distortions, image modifications, or any combination thereof.

17. The non-transitory computer-readable medium of claim 13, wherein detecting the anomalies based on at least the features of the dental image comprises determining whether the dental image includes fraudulent manipulations.

18. The non-transitory computer-readable medium of claim 13, wherein the dental information of the patient can include patient profile information including information associated with patient gender identification, patient age, previously claimed images associated with the patient, or any combination thereof.

19. The non-transitory computer-readable medium of claim 13, further comprising:
instructions for matching the features of the dental image associated with the patient with dental information associated with an additional patient.

20. The non-transitory computer-readable medium of claim 13, further comprising:
instructions for matching the features of the dental image associated with the patient with features of an additional dental image associated with the patient or an additional patient.
